(19) United States
(12) Patent Application Publication
(10) Pub. No.: US 2012/0296150 A1
(43) Pub. Date: Nov. 22, 2012

(54) PORTABLE APPARATUS FOR LOCAL COMBINED ELECTROMAGNETIC IRRIDATION

(76) Inventors: Sergey V. Pletnev, Minsk (BY); Andrey S. Pletnev, Minsk (BY); Anatoly A. Barsukov, Minsk (BY); Sergey V. Pletnev, Minsk (BY)

(21) Appl. No.: 13/522,934

(22) PCT Filed: Jan. 19, 2010
(86) PCT No.: PCT/IB10/50235

§ 371 Date: Jul. 18, 2012

(51) Int. Cl. A61N 2/02; A61N 5/06

(52) U.S. Cl. 600/13

(57) ABSTRACT

The present invention relates to the electromagnetic field physiotherapy and may be used for treating and preventing various diseases and disorders.

A portable apparatus for local integrated electromagnetic irradiation comprises a device for generating a light field with a combinable wavelength, an array of individual light sources with different wavelengths, a controllable magnetic field source including at least one coil and a core made of a ferromagnetic material and a device for controlling and combining the light and magnetic fields. The apparatus is mainly characterized in that the core of the magnetic field source is made magnetically closed from a rear side and is provided with a clearance in the region spatially combined with the light field, with the magnetic field controlling device being capable of generating a low-frequency pulsed magnetic field in the combined spatial region with an adjustable frequency in the range of 1-200 Hz and an amplitude in the range of 10-30 mT and having controlling inputs coupled to the controlling and combining device.

### FIELD OF INVENTION

The present invention relates to the electromagnetic field physiotherapy and may be used for treating and preventing various diseases and disorders.

### PRIOR ART

RF Patent No. 2090224 has provided a physiotherapeutic apparatus comprising a power supply unit, a series of light-emitting diodes (LED) with different light and infrared spectral ranges forming a LED array and also a means to control LED emissivity characterized in that light-emitting diodes of this series are connected therein to form at least one series electrical circuit coupled to the power supply unit via the control means.

This apparatus has a wide spectrum of light exposure on injured tissues, however, in the limited surface area and does not penetrate deeply into muscle tissues.

The DE Patent No. 10332771 provides a portable radiating apparatus for using electromagnetic radiation with wavelength of light emitted by a controllable light field source with individual light sources of different spectra which are combined in groups. The apparatus also comprises a controllable magnetic field source. The light and magnetic field are emitted by groups of pulses with a frequency of 20 and 1,200 pulses/second within preset time intervals.

The magnetic field more deeply penetrates into tissues and provides an efficient therapeutic effect thereon.

A combination of magnetic field and light irradiation increases efficiency and depth of penetration of optical radiation, however, it is not possible to achieve efficient values of the magnetic field in the exposure area due to the use of a coreless induction coil in the prototype.

The DE Patent Application No. 3101715 provides a device in which an array of electric magnets with ferromagnetic cores is disposed on the backside of the LED array. Such magnets allow higher magnetic field parameters to be achieved.

Magnets, however, are disposed behind the light-emitting diodes and the magnetic field substantially reduces in the area of the light field action, since the maximal magnetic field intensity is spaced from a target object at a distance defined by thickness of the LED array. Therefore, maximum efficiency of the curative effect is not achieved. In addition, the use of separated cores results in substantial energy dissipation of the field on the back side of cores and prevents achieving effective values of the magnetic field with maximal energy efficiency.

Object of the invention is to enhance curative effect, expand functional possibilities and range of application, provide a portable energy-efficient device suitable for in-hospital and home use.

### Spirit of the Invention

The object of the invention is achieved as described hereinafter.

### Variant 1

A portable apparatus for local integrated electromagnetic irradiation comprising a device for generating a light field with a combinable wavelength including an array of individual light sources with different wavelengths, a controllable magnetic field source including at least one coil and a core generating an external magnetic field combined with the light field in a specific area of space and a device for controlling and combining the light and magnetic fields. The apparatus is characterized in that the core of the magnetic field source is made of a ferromagnetic material, magnetically closed from a rear side and is provided with a clearance in the region spatially combined with the light field, with the magnetic field controlling device being capable of generating a low-frequency pulsed magnetic field in the combined spatial region with an adjustable frequency in the range of 1-200 Hz and an amplitude in the range of 10-30 mT and having controlling inputs coupled to the controlling and combining device. The array of individual light sources is disposed in the area between opened core poles to spatially combine the light and magnetic fields in the external zone facing the exposure area. Such a configuration of the core and a relative arrangement of the light-source array provide most efficient action on areas to be treated with minimal power consumption and compact dimensions.

Most preferably, the core is made as a U-shaped magnet with a gap facing the irradiated area, while the array of individual light sources is arranged between open core poles.

### Variant 2

According to the other embodiment of the present invention, a portable apparatus for local integrated electromagnetic irradiation comprising a device for generating a light field with a combinable wavelength includes a set of individual light sources with different wavelengths, a controllable magnetic field source including at least one coil and a ferromagnetic core generating an external magnetic field, a means for spatially combining the light and magnetic fields and a device for controlling and combining the light and magnetic fields. This embodiment is characterized in that the cores of the magnetic field source are made magnetically closed from the rear side and have a clearance in the area spatially combined with the light field, with the magnetic field controlling device being capable of generating a low-frequency pulsed magnetic field in the combined spatial region with an adjustable frequency in the range of 1-200 Hz and an amplitude in the range of 10-30 mT and having controlling inputs coupled to the controlling and combining device, with the means for combining optical and magnetic fields being made in the form of a light guide transmitting light from the set of optical radiation sources to the region spatially combined with an effective magnetic field and facing the exposure area. In this case, the set of optical radiation sources may be arranged outside the gap of pole pieces and as a result, this set may be significantly expanded both in terms of spectral range and in terms of higher total power, since the number and spectral range of light sources are not limited by space in the magnet gap in such a configuration.

Further, the means for combining optical and magnetic fields may comprise directional reflectors and the means for mixing combinable optical radiation made in the form of planar or fiber light guides and reflectors.

### LIST OF DRAWINGS

FIG. 1—block diagram of a control device.

FIG. 2—an embodiment of the apparatus having light sources arranged in the gap of the magnet U-shaped core.

FIG. 3—an embodiment of the apparatus having light sources arranged outside the magnet U-shaped core, a light guide and reflectors.

### Specific Embodiments of the Invention

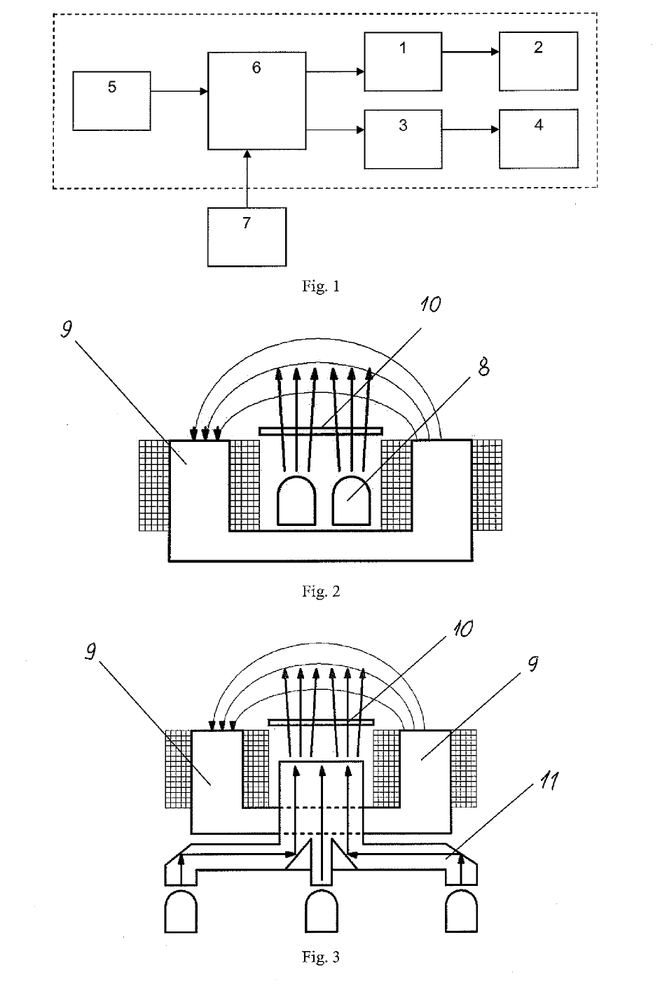

Referring to FIG. 1, a light-and-magnetotherapy apparatus comprises a portable housing adapted to dispose therein as follows: a generator 1 for generating a current of the preset intensity and form to supply power to a magnetic-exposure inductor 2 and a control unit 3 of a light source 4 of the visible optical spectrum.

Control buttons 5 disposed on the housing are adapted to control the apparatus and set the parameters such as magnetic exposure strength, selection of color of the optical radiation and intensity thereof. In general, the apparatus is controlled by a microprocessor controller 6 which also performs combining functions. The apparatus is powered by an external network adapter 7.

As shown in FIG. 2, structurally an optical radiation source 8 is arranged between poles of the magnetic-exposure inductor 9 to locally expose a specific area of the patient's biological tissue to a combination of a magnetic field and light exposure the efficiency of which increases due to the magnetic field effect. In addition, a polarizer 10 is mounted on the path of light propagation behind which the light is propagated in parallel planes, thereby providing more expressed therapeutic effect.

The polarizer 10 converts light propagating in all directions into plane-polarized light.

In the second embodiment shown in FIG. 3, structurally the optical radiation source 8 may be disposed in the housing outside the working gap of the inductor, for example, along the periphery or in other part of the housing. In this case, the light flux from the light source 4 is transmitted via a planar light guide with reflectors 11 to combine it with an exposure region of the magnetic field. Such a light guide may be made of an optically transparent material with a reflective coating or with total internal reflection. The light guide may be also made of an optical fiber. This embodiment of the light source arrangement substantially increases therapy efficiency by expanding the range of spectral characteristics through the use of a larger number of groups of light guides with different spectral characteristics since their arrangement and space are not limited by the gap between polar tips. It is noteworthy, the most intensive light flux may be obtained in the region of the maximal action of the magnetic field due to transmission of light via light guides. Such a structure may also have technological advantages since it allows light guides to be arranged on a printed board jointly with other electronic units. This also increases service reliability in different conditions.

A portable light-and-magnetotherapy apparatus has been produced for treatment of an extensive range of disorders by the pulsed magnetic field (magnetotherapy) and polarized optical radiation of the visible spectrum (phototherapy).

### Main Specifications

### Overall Dimensions of the Apparatus

| | |
|---|---|
| Length | 200 mm |
| Width | 94 mm |
| Height | 40 mm |

Weight, without a power supply unit, not more than 0.35 kg

### Magnetic Exposure

| | | |
|---|---|---|
| Pulse packet repetition rate | 1-10 +/− 1 | Hz |
| Signal frequency in pulse packet | 40-200 | Hz |
| Maximal pulsed magnetic induction | 30 | mT |
| Exposure intensity adjustment: | | |
| min | 10 +/− 5 | mT |
| max | 25 +/− 5 | mT |

### Optical Exposure

Light source—semiconductor light-emitting diodes.

### Optical Radiation Wavelength

| | |
|---|---|
| Blue color | 465-475 nm |
| Green color | 515-535 nm |
| Yellow color | 585-595 nm |
| Red color | 610-635 nm |
| Infrared | 920-960 nm |

### Light Intensity Adjustment

Three subbands: 50, 75 and 100% of maximal value.

A polarizing light filter converts non-polarized light into polarized light.

### Possible Combination Modes

1. Exposure to pulsed magnetic field together with steady light of preselected color.
2. Exposure to pulsed magnetic field simultaneously with pulsed light of preselected color.
3. Exposure to pulsed light of preselected color in intervals between pulse packets of magnetic exposure.
4. Exposure to pulsed light of preselected set of colors in different combinations of exposure to pulsed magnetic field.

It should be noted that functionally light and magnetic exposure may be used separately.

A combination of new features of the apparatus and efficiency thereof allow it to be used for treatment of an extensive range of disorders. A flexible system of adjustment and possible combinations makes it possible not only to develop new techniques for treatment of various disorders, but also individualize exposure for specific patients as well as to use it not only for treatment, but also for prevention of diseases, in restorative medicine, sport sphere, etc.

The apparatus structure allows for using it both under in-hospital and in-home conditions. Therefore, this expands the range of apparatus application.

Tests of the apparatus have demonstrated high efficiency thereof for treating and preventing an extensive range of skin and annexa diseases, for treating traumas, concussions and in stomatology.

1. A portable apparatus for local combined electromagnetic irradiation comprising:
a device for generating a light field with a combinable wavelength including an array of individual light sources with different wavelengths,
a controllable magnetic field source including at least one coil and a core made of a ferromagnetic material generating an external magnetic field combined with the light field in a specific area of space; and
a device for controlling and combining the light and magnetic fields, presenting a core of the magnetic field source made magnetically closed from a rear side and provided with a clearance in the region spatially combined with the light field, with the device for controlling magnetic field is capable of generating a low-frequency pulsed magnetic field in the combined spatial region with an adjustable frequency in the range of 1-200 Hz and an amplitude in the range of 10-30 mT and having controlling inputs coupled to the device for controlling and combining magnetic fields, with the array of individual light sources being disposed in the area between opened cores to spatially combine the light and magnetic fields in the external zone facing the exposure area.

2. The apparatus as set forth in claim 1, wherein the core is made as a U-shaped magnet with a gap facing the irradiated area, while the array of individual light sources is arranged between open core poles.

3. A portable apparatus for local combined electromagnetic irradiation comprising:
a device for generating a light field with a combinable wavelength including a set of individual light sources with different wavelengths,
a controllable magnetic field source including at least one coil and a ferromagnetic core generating an external magnetic field, a means for spatially combining the light and magnetic fields; and
a device for controlling and combining the light and magnetic fields presenting a core of the magnetic field source made magnetically closed from the rear side and has a clearance in the area spatially combined with the light field, with the device magnetic field controlling device being capable of generating a low-frequency pulsed magnetic field in the combined spatial region with an adjustable frequency in the range of 1-200 Hz and an amplitude in the range of 10-30 mT and having controlling inputs coupled to the controlling and combining device, with the device for generating the light field including a means for combining optical and magnetic fields being made in the form of light guides transmitting light from the set of optical radiation sources to the region spatially combined with an effective magnetic field and facing the exposure area.

4. The apparatus as set forth in claim 3 wherein the means for combining optical and magnetic fields may comprise directional reflectors and the means for mixing combinable optical radiation.
